(19) United States
(12) Patent Application Publication
(10) Pub. No.: US 2013/0268051 A1
(43) Pub. Date: Oct. 10, 2013

(54) STENT DELIVERY SYSTEM

(71) Applicants: David Atlani, Jouy Le Moutier (FR); Hikmat Hojeibane, Princetown, NJ (US); Gonzague Issenmann, Clichy (FR)

(72) Inventors: David Atlani, Jouy Le Moutier (FR); Hikmat Hojeibane, Princetown, NJ (US); Gonzague Issenmann, Clichy (FR)

(73) Assignee: STENTYS S.A.S., PARIS (FR)

(21) Appl. No.: 13/994,968

(22) PCT Filed: Jan. 30, 2012
(86) PCT No.: PCT/IB2012/050403

§ 371 Date: Jun. 17, 2013

Related U.S. Application Data
(60) Provisional application No. 61/438,477, filed on Feb. 1, 2011.

(30) Foreign Application Priority Data
Feb. 1, 2011 (FR) 11 50788

(51) Int. Cl. A61F 2/966

(52) U.S. Cl. CPC A61F 2/966
USPC 623/1.11

(57) ABSTRACT

A system includes: a stent having a proximal end; a support including the stent engaged thereon, having a longitudinal axis and being equipped with a stop against which the proximal end is applied; a sheath having a distal end, which can slide relative to the support and stop, between a distal overlap position of the stent and a proximal release position for deploying the stent, and a first inflatable balloon situated between the support and stent, including a distal portion that protrudes past the distal end, on the distal side thereof when the latter is in the distal position, and having, on the distal side, a transverse section larger than or equal to that of the sheath, the distal end situated back from the distal end in the proximal direction, and situated opposite the distal portion, such that the distal portion protrudes partially past the distal end of the sheath.

The present invention relates to a stent delivery system.

To reestablish the caliber of a bodily conduit, it is well known to place, in that bodily conduit, a deployable tubular framework with an openwork structure, commonly called a “stent.” Such a stent can adopt a radially contracted state, allowing it to be inserted into the bodily conduit, and a radially expanded state, in which it is found when it is implanted and owing to which it reestablishes the caliber of the bodily conduit. The stent can be made from an elastic material making it self-expanding, or can be made from a material making it necessary to perform, in whole or in part, a forced expansion (often called “stent on balloon”).

One well-known system for bringing a self-expanding stent to its implantation site and delivering that stent to the site consists of a catheter inserted into the patient and slid into the latter's bodily conduit. This sliding is done until the distal end of the catheter, which comprises the stent in the aforementioned contracted state, is found at the implantation site. Such a catheter comprises a central support comprising the stent on its distal end, and a sheath capable of being slid relative to said central support so as to free the expansion of the stent.

It will be understood that the terms “distal” and “proximal” used in this description should be considered relative to the operator, “distal” meaning remote from the operator (or on the contrary close to the implantation site) and “proximal” meaning close to the operator (or on the contrary remote from the implantation site).

Another well-known system for bringing a stent on balloon toward its implantation site is a catheter comprising an inflatable balloon on which the stent is placed, this balloon making it possible to perform said forced expansion.

The positioning of the stent in the bodily conduit to be treated must be precise, failing which the recalibration of that conduit is ineffective or partially effective. This positioning must be even more precise inasmuch as the stent is no longer movable once it is expanded and bearing against the wall of the bodily conduit. In the case of a self-expanding stent placed using a known catheter, the precise positioning of the stent depends greatly on the dexterity and experience of the operator, since the removal of the sheath is usually done by applying a relative movement between said support and the sheath, involving exerting a pulling force on the sheath; given that this action is done from outside the patient and the catheter itself is inserted into a guide catheter, it tends to cause an uncontrolled movement of the sheath and the catheters during the deployment phase of the stent. Furthermore, the stent being elastically deformed when it is radially contracted, there is a risk of ejection of the stent relative to the catheter during the release of that stent, leading the latter to “jump” from the catheter after a certain degree of removal of the sheath.

The present invention aims to resolve all of these essential drawbacks, by providing a system for delivering a self-expanding stent or a “stent on balloon” that allows precise positioning of the stent in the bodily conduit to be treated.

Another problem that arises with a stent on balloon comprising a coating constituting a medication is that the forward movement of the catheter through the conduit to be treated causes the risk of damaging said coating by friction against the wall of the conduit. In certain anatomies, the wall of the conduit can also be damaged by the friction of the stent against it.

The present invention also aims to resolve these drawbacks, by providing a system for delivering a stent on balloon that makes it possible to avoid contact between the surface of the stent and the bodily conduit to be treated until the stent is deployed.

Document US 2002/052640 describes a system for delivering a stent including a sheath or biocompatible or bio-absorbable filaments for setting the stent. The sheath has a sufficient strength to keep the stent in the contracted state, but is fragile enough to be broken or expanded by inflating the balloon. Such a system, with a non-sliding sheath, does not correspond to the system according to the invention, in which the sheath can slide and in which the aforementioned problem arises of precisely positioning the stent in the bodily conduit to be treated.

Documents WO 2009/005933, US 2005/0288763, US 2007/0088368 or U.S. Pat. No. 5,456,694 describe various other existing systems, also not making it possible to resolve the aforementioned problems.

According to the invention, the system comprises:

a stent having a proximal end;

a support comprising the stent engaged thereon, having a longitudinal axis and being equipped with a stop against which the proximal end of the stent is applied;

a sheath having a distal end, which can be slid relative to said support and said stop, between a distal overlap position of the stent and a proximal release position for the deployment of the stent, and

a first inflatable balloon situated between said support and the stent, comprising a distal portion that protrudes past the distal end of the sheath, on the distal side thereof, when the latter is in said distal position, and having, in the inflated state, over all the length thereof, or over a part of the length thereof, on the distal side, a transverse section larger than or equal to that of the sheath,

the distal end of the sheath being, in said distal overlap position of the stent, situated back from said distal end, along said longitudinal axis of the support, in the proximal direction, and being situated opposite said distal portion, such that said distal portion protrudes partially past said distal end of the sheath, on the distal side of said end.

Thus, to deliver the stent, said first balloon is first supplied with inflating fluid, which first inflates said distal portion of the balloon and then creates an inflation wave moving in the proximal direction; this inflation wave causes the balloon to come into contact with the distal end of the sheath, then sliding of the sheath along the wall of the balloon, which makes it possible to remove the sheath; at the beginning of that removal, the distal end of the stent is maintained by said inflated distal portion of the balloon, which prevents any risk of the stent “jumping” relative to said support. During this inflation of the balloon and removal of the sheath, the bearing of the proximal end of the stent against said stop results preponderantly in avoiding any risk of removal of the stent at the same time as the sheath.

The removal of the sheath is therefore done without any pulling force having been exerted on said sheath from outside the patient, which avoids the risk of moving the entire system, and therefore the stent, relative to the implantation site. On the contrary, the inflation of said distal portion of the balloon immediately presses the distal end of the stent against the wall of the bodily conduit as of the beginning of removal of the sheath, and therefore performs a certain axial immobilization of the stent relative to said conduit as of that beginning of removal.

As a result, the system according to the invention makes it possible to deliver the stent in a very precise position.

Preferably, the system includes locking means that can be actuated between a locking position and an unlocking position, these locking means being able, in the locking position, to immobilize the sheath in said distal overlap position and, in the unlocking position, to release the sliding of the sheath.

These means are in the locking position during the insertion and sliding of the system in the bodily conduit toward the implantation site, and are actuated in the unlocking position of said sheath when the stent is located opposite said implantation site.

These locking means can comprise a sheath having a length such that it extends outside the patient when the stent is located opposite the implantation site, and means for immobilizing the sheath relative to said support, capable of being controlled from outside the patient.

According to one preferred embodiment of the invention, however, these locking means comprise a second inflatable balloon, fixed in position relative to said support and situated inside the sheath, the inflation of which corresponds to said locking position and the deflation of which corresponds to said unlocking position; in the inflated state, this second balloon is pressed against the inner surface of the sheath so as to generate friction therewith, immobilizing the latter in the axial direction relative to said support, and, in the deflated state, this second balloon is not pressed against the inner surface of the sheath so as to allow the sliding of the latter.

According to another embodiment of the invention, said locking means also comprise a second inflatable balloon fixed in position relative to said support and the inflation of which corresponds to said locking position while the deflation thereof corresponds to said unlocking position; in this case, this second balloon is situated outside the sheath, on the proximal side of a proximal end of said sheath; the second balloon has, in the inflated state, a diameter larger than that of the sheath, so that this second balloon forms a stop against which the proximal end of the sheath bears so as to block the sliding of the sheath; in the deflated state, this second balloon has a diameter smaller than that of the sheath, so that this second balloon does not form a stop for said proximal end of the sheath and therefore does not hinder the sliding of the sheath.

This second balloon is therefore in the inflated state during the insertion and sliding of the system in the bodily conduit of the patient toward the implantation site, thereby locking the sheath in said distal overlap position, and is deflated just before the deployment operation of the stent, so as to release the removal of the sheath.

Said second balloon can in particular be situated close to said stop, on the proximal side thereof.

The distance separating the distal end of said balloon and the distal end of the sheath when the latter is in the distal position, must be greater than 0.5 times the diameter of the sheath; it is preferably comprised between one and two times the diameter of the sheath.

Said first balloon can have a cylindrical shape, with a diameter at least equal to that of the sheath; it can also have a conical shape, whereof the distal portion, with the larger diameter, has a diameter at least equal to that of the sheath; it can also have one or more longitudinal ribs, in particular four equidistant longitudinal ribs, so as to decrease any friction with the sheath; it can also have, in the deflated or folded state, a distal portion having a transverse section larger than that of its proximal portion, this distal portion in particular being able to have a more or less ovoid or hernial shape; it can also have a helical groove in the inflated state, the purpose of which is to reduce the force radially exerted by the balloon on the sheath as well as the friction existing between said balloon and the stent.

This first balloon can also have a shape other than a circular shape in transverse section, for example oval, so as to decrease any friction with the sheath.

According to one possibility, said support comprises a widened distal end piece (commonly called “tip”), capable of favoring the progression of the system in a bodily conduit, and the sheath comprises a sleeve extending its distal end, which is dimensioned so as to extend to said end piece or tip, this sleeve being made from a material more flexible than that of the sheath, not offering resistance to the inflation of said distal portion of said first balloon, or a low resistance to that inflation.

This sleeve thereby allows the system to have a substantially smooth and continuous outer wall between said end piece and said sheath.

According to one possible embodiment of the invention, said support comprises a widened distal end piece, capable of favoring the progression of the system in a bodily conduit, and said distal portion of the first balloon is shaped so as to surround a proximal portion of that end piece and, in the non-inflated state, to extend close to the distal end of the sheath.

Said first balloon, thus shaped, also allows the system to have a substantially smooth and continuous outer wall between said end piece and said sheath.

Preferably, the system includes a proximal inflation restricting element, making it possible to ensure that the inflation of said first balloon starts at said distal portion of the balloon and progresses in the proximal direction.

This restriction element makes it possible to prevent early proximal inflation from occurring, i.e. proximal inflation occurring at the same time as the distal inflation, which would produce a radial force on the sheath, generating tightening thereof capable of hindering, or even preventing, the removal of the sheath.

According to one possible embodiment of the invention, this restriction element is in the form of a sleeve surrounding said balloon in an adjusted manner, one distal end of said sleeve protruding past the distal end of the sheath and being situated back from the distal end of said first balloon in the proximal direction, and this sleeve being longitudinally tearable under the effect of the inflation of this first balloon, so as not to hinder the inflation.

The inflation of said first balloon is thus done from said distal end of this first balloon and progresses in the proximal direction, the proximal portion of said first balloon not being able to inflate, being maintained by the sleeve; this inflation causes the gradual tearing of the sleeve from said distal end of the sleeve, which allows the inflation to continue, in the proximal direction.

Said sleeve can be made from a material with a low coefficient of friction, in particular PTFE (polytetrafluoroethylene). This material favors the engagement of the sleeve on the stent during the assembly of the device, and the removal of said first balloon relative to the stent once the latter is placed.

Said sleeve can in particular comprise a longitudinal notch arranged from its distal end, constituting a beginning of longitudinal tearing.

Preferably, the system includes an element for preventing or restricting the radial expansion of the sheath, also resulting in preventing early proximal inflation of said first balloon from occurring.

According to one possible embodiment of the invention, this expansion preventing or restricting element comprises a sleeve made from an elastically deformable material, extending said distal end of the sheath, having, in the non-inflated state of said first balloon, a conical shape converging radially, i.e. extending from said distal end of the sheath to said first balloon, and expanding radially during the inflation of said first balloon, to adopt a flared shape, radially divergent.

This radial expansion generates a radial elastic return force of the material making up said sleeve, favorable to obtaining an effective removal movement of the sheath.

This expansion preventing or restricting element can also comprise a ring made from a material not radially stretchable, fixed at the distal end of the sheath. This ring can be made from a material with a low coefficient of friction, for example a metal, for the same reasons as indicated above. The ring can also form a radiopaque element.

The invention will be well understood, and other features and advantages thereof will appear, in reference to the appended diagrammatic drawing, showing, as non-limiting examples, several possible embodiments of the concerned delivery system.

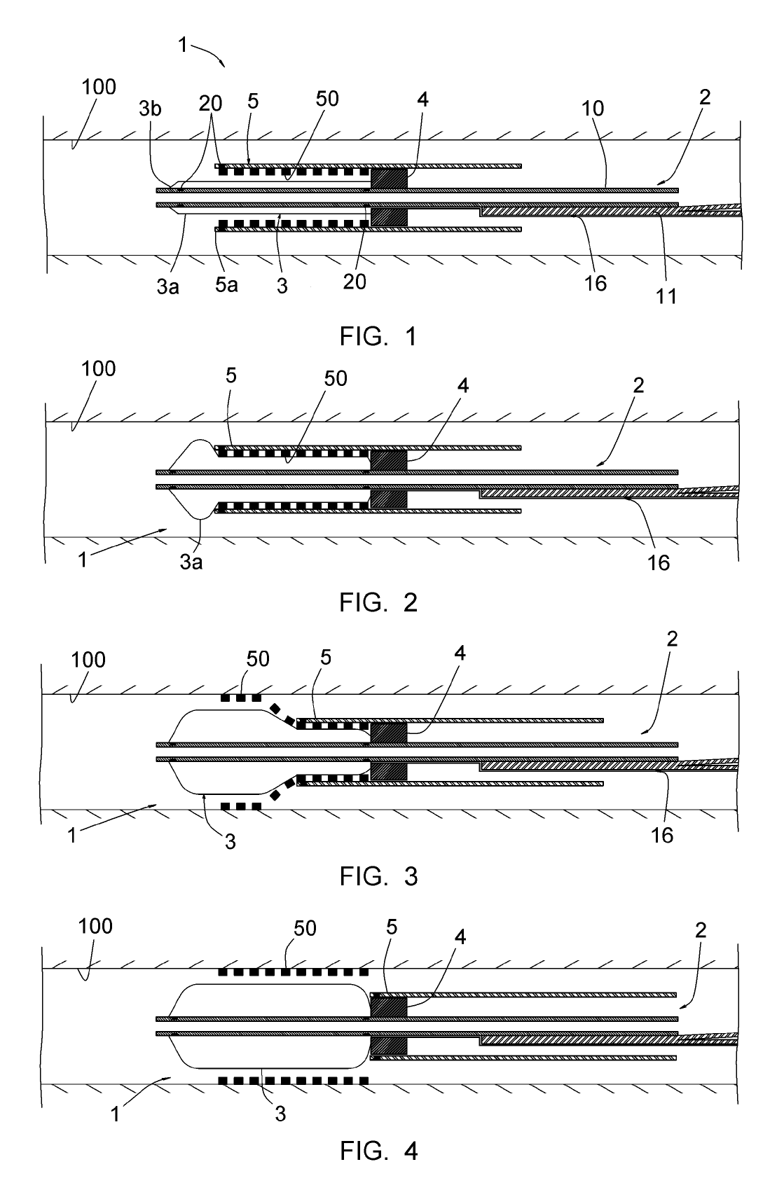

FIG. 1 is a view in longitudinal cross-section according to a first embodiment, when it is engaged in a bodily conduit, before deployment of the stent comprised by that system;

FIG. 2 is a view similar to FIG. 1, at the beginning of the deployment procedure of the stent;

FIG. 3 is a view similar to FIG. 1, during the deployment procedure of the stent;

FIG. 4 is a view similar to FIG. 1, at the end of the procedure for deploying the stent;

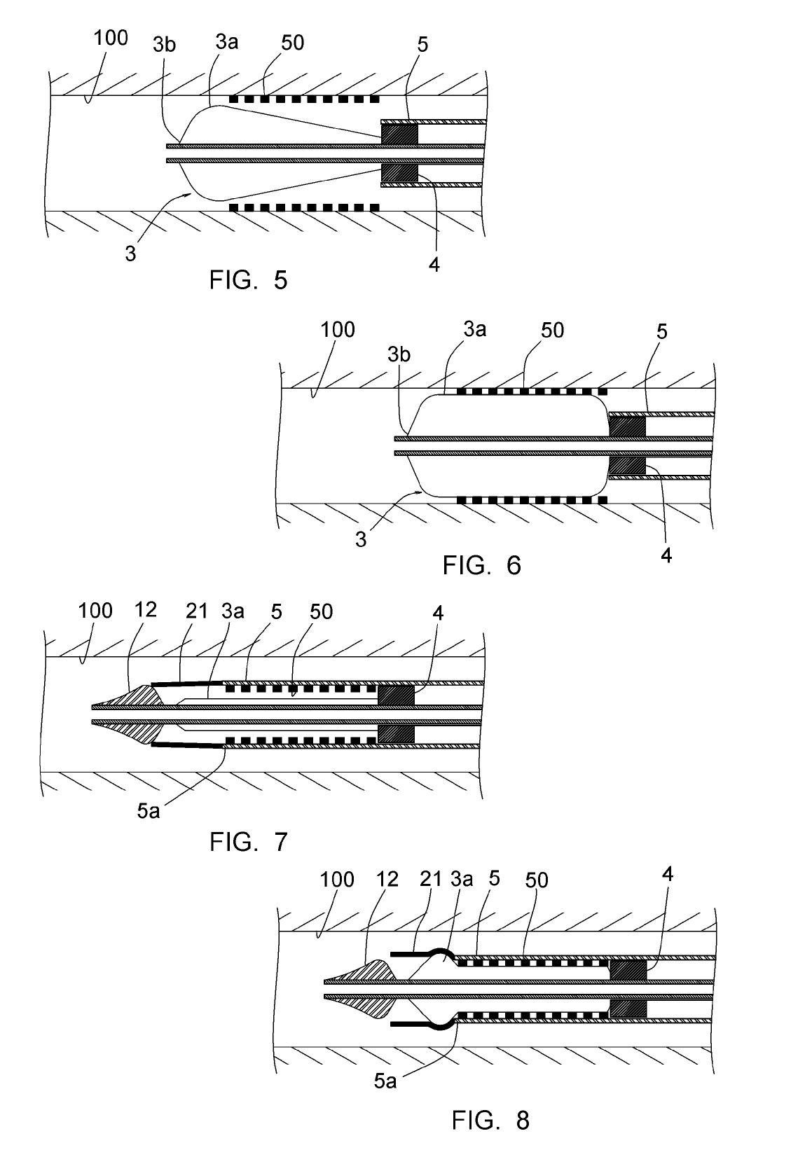

FIG. 5 is a view similar to FIG. 4, according to another embodiment;

FIG. 6 is a view similar to FIG. 4, according to still another embodiment;

FIG. 7 is a cross-sectional view similar to FIG. 1, according to still another embodiment;

FIG. 8 is a view similar to FIG. 7 of this same embodiment, at the beginning of the procedure for deploying the stent;

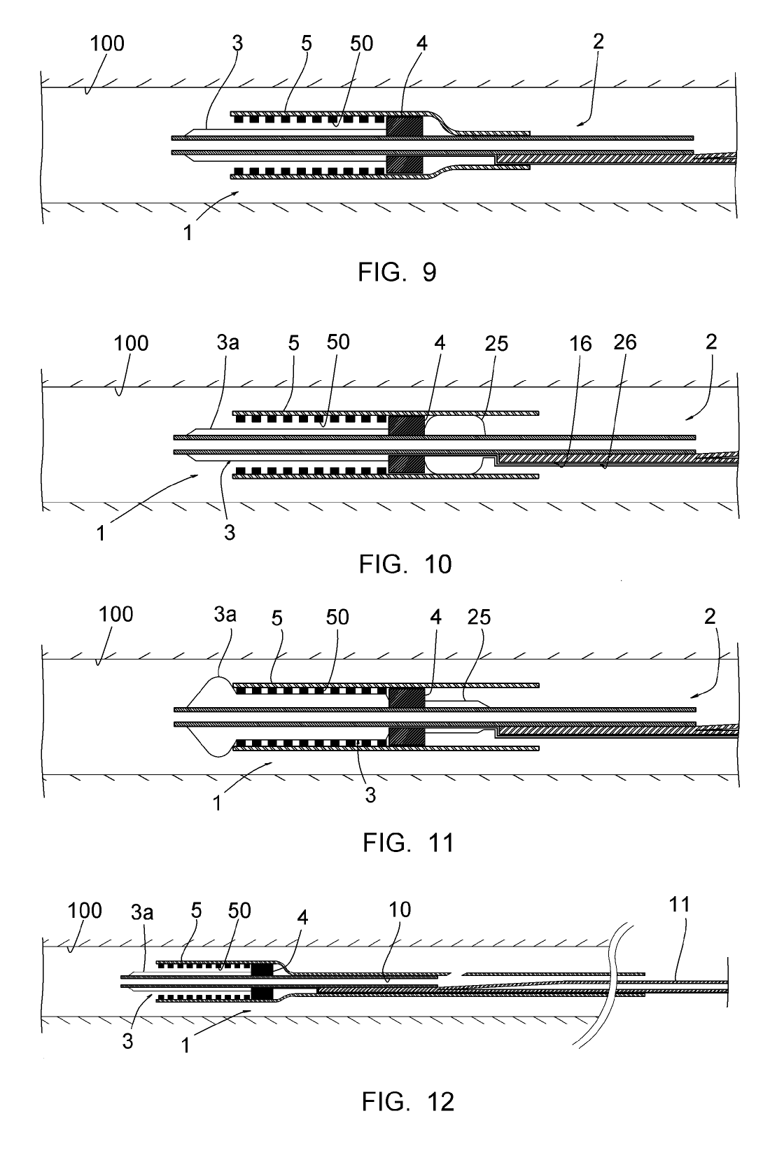

FIG. 9 is a cross-sectional view of the system similar to FIG. 1, according to still another embodiment;

FIG. 10 is a cross-sectional view similar to FIG. 1, according to still another embodiment;

FIG. 11 is a cross-sectional view of this same embodiment, at the beginning of the procedure for deploying the stent;

FIG. 12 is a cross-sectional view of the system similar to FIG. 1, according to still another embodiment;

FIGS. 13 to 15 are transverse cross-sectional views thereof, according to three possible embodiments of a balloon comprised by said system;

FIG. 16 is a cross-sectional view of the system similar to FIG. 1, according to still another embodiment;

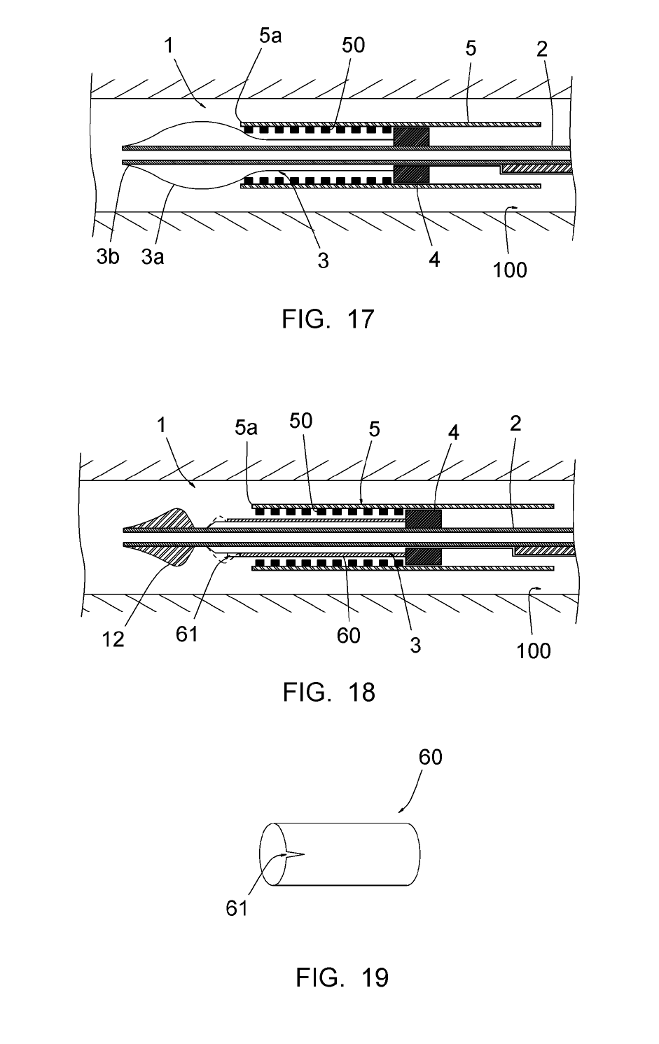

FIG. 17 is a cross-sectional view of the system similar to FIG. 1, according to still another embodiment;

FIG. 18 is a cross-sectional view of the system similar to FIG. 1, according to still another embodiment;

FIG. 19 is a perspective view of a sliding element in the form of a sleeve, comprised by the system shown in FIG. 18;

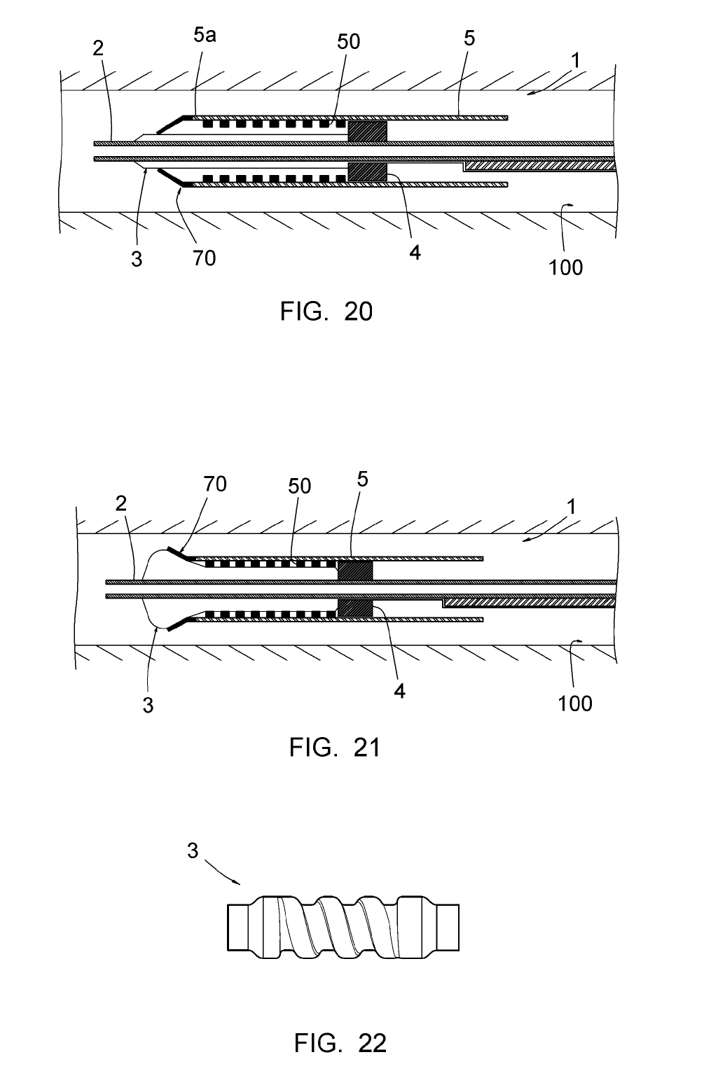

FIG. 20 is a cross-sectional view of the system similar to FIG. 1, according to still another embodiment, in the uninflated state of a balloon comprised by said system;

FIG. 21 is a view of the system shown in FIG. 20, at the beginning of inflation of said balloon, and

FIG. 22 is a side view of another embodiment of said balloon.

For simplification, the parts or elements of one embodiment that are found identically or similarly in another embodiment will be designated using the same numerical references.

FIGS. 1 to 4 show a system 1 for delivering a stent 50, engaged in a bodily conduit 100.

The stent 50 is, as is well known, intended to reestablish the caliber of the bodily conduit 100, particularly at a stenosis formed by atheromatous plaques (not shown). It is formed by a deployable tubular framework, with an mesh structure, which can adopt either the radially contracted state shown in FIG. 1, which allows it to be inserted into the bodily conduit 100, or the radially expanded state shown in FIG. 4, in which it is implanted and reestablishes the caliber of the bodily conduit 100. The stent 50 can be made from an elastic material making it self-expanding, for example an alloy of nickel and titanium, or can be made from a material making it necessary to perform, in whole or in part, a forced expansion.

The system 1 comprises an inner support 2 comprising the stent 50, an inflatable balloon 3, a stop 4 and an outer sheath 5.

The inner support 2 comprises a distal tubular portion 10 connected to a proximal portion 11.

The distal portion 10 comprises the balloon 3 and the stop 4, fixed thereto.

The proximal portion 11 has a sufficient length to protrude to the outside of the patient when the distal portion 10 is found at the implantation site of the stent 50, and thus makes it possible to move the system 1 relative to the bodily conduit 100.

The balloon 3 is formed by a flexible enclosure engaged around a distal part of the distal portion 10. On the distal side, this flexible enclosure is sealably fixed to the distal portion 10, on the entire periphery of said distal portion, at one end 3b; on the proximal side, the flexible enclosure is fixed to a radially inner zone of the distal surface of the stop 4, as shown, or to the distal portion 10, near said stop. In the inflated state, the balloon 3 has a transverse section larger than that of the sheath 5; in the example embodiment shown in FIGS. 1 to 4, it is cylindrical, and has a transverse section smaller than that of the bodily conduit 100.

The balloon 3 has a length greater than that of the stent 50 and thus comprises a distal portion 3a, adjacent to the distal end 3b, which protrudes past the distal end 5a of the sheath 5, on the distal side thereof, as particularly visible in FIG. 1.

This balloon 3 is supplied with pressurized fluid from outside the patient, through a conduit 16 running along the support 2 and passing between the distal portion 10 and the stop 4.

The latter is formed by a thick washer secured to the distal tubular portion 10, the diameter of which is equal to or slightly larger than the inner diameter of the sheath 5, so as to allow sliding of that sheath 5 thereon but with friction. The proximal end of the stent 50 bears against the distal surface of said stop 4.

The sheath 5 is engaged around the stent 50, whereof it ensures, in the distal position shown in FIG. 1, the maintenance in the radially contracted state. It is engaged on the stop 4 and, in the illustrated example, extends over the proximal side of said stop 4, over a length in the vicinity of two thirds that of the stent 50. This sheath 5 therefore does not extend along the proximal portion 11 of the support 2 and is not intended to be slidingly actuated from outside the patient, like a traditional counterpart sheath, but to be actuated by inflating the balloon 3, as described below.

It appears that, in the distal position of the sheath 5, overlapping the stent 50, the distal end 5a of the sheath 5 is situated back from said distal end 3b, along said longitudinal axis of the support 2, in the proximal direction, and is situated opposite said distal portion 3a, so that this distal portion 3a protrudes partially from said distal end 5a of the sheath 5, on the distal side of said end.

In practice, the system 1 is inserted and is slid in the bodily conduit 100 in the state shown in FIG. 1. The maintenance of the sheath 5 in the distal overlapping position of the stent 50 is ensured owing to the friction existing between the stop 4 and the sheath 5.

The arrival of the distal portion of the system 1 at the implantation site is detected using radiopaque markers 20 comprised by the support 2 and the sheath 5, in particular, in the example shown in FIG. 1, a marker 20 situated on the portion 10 at the distal end 3b of the balloon 3, a marker 20 situated on the portion 10 at the proximal end of the balloon 3, and a marker 20 situated at the distal end of the sheath 5.

The balloon 3 is then supplied with inflating fluid, which first inflates the distal portion 3a thereof and then creates an inflation wave moving in the proximal direction. This inflation wave causes the balloon 3 to come into contact with the distal end 5a of the sheath 5 (see FIG. 2), then causes that sheath to slide along the wall of said balloon 3, which makes it possible to remove the sheath 5 and therefore to gradually release the stent 50 (see FIGS. 3 and 4).

At the beginning of this removal, the distal end of the stent 50 is maintained by the distal portion 3a of the balloon 3, which prevents any risk of “jumping” of the stent 50 relative to the support 2. The bearing of the proximal end of the stent 50 against the stop 4 prevents the risk of removal of the stent 50 at the same time as the sheath 5.

The removal of the sheath 5 is therefore done without any pulling force having been exerted on this sheath from outside the patient, which prevents the risk of movement of the system 1, and therefore of the stent 50, relative to the implantation site. On the contrary, the inflation of the distal portion 3a of the balloon immediately presses the distal end of the stent 50 against the wall of the bodily conduit 100 as of the beginning of removal of the sheath 5, and therefore performs a certain axial immobilization of that stent 50 relative to said conduit 100 as of this beginning of removal.

FIG. 5 shows another embodiment of the system 1, in which the balloon 3 is conical, with the exception of its distal surface, which is rounded.

FIG. 6 shows an embodiment of the system 1 that is practically identical to that previously described, except that the balloon 3 has a larger diameter, slightly smaller than that of the bodily conduit 100. Such a balloon 3 is used in particular when it involves performing a forced deployment of the stent 50.

FIGS. 7 and 8 show another embodiment of the system 1, in which said distal portion 10 comprises, at its distal end, a widened and rounded end piece 12 (commonly called “tip”) and the sheath 5 comprises a sleeve 21 extending its distal end, which is dimensioned so as to extend as far as the end piece or tip 12. This sleeve 21 is made from a material more flexible than that of the sheath 5, not offering resistance to the inflation of the distal portion 3a of the balloon 3. The end piece 12 favors the insertion and sliding of the system 1 in the conduit 100, and the sleeve 21 allows that system to have a substantially smooth and continuous outer wall between the end piece 12 and the sheath 5 (see FIG. 7).

FIG. 9 shows another embodiment of the system 1, in which the sheath 5 bends on the proximal side, beyond the stop 4, and is connected to the support 2.

This connection favors the maintenance of the sheath 5 relative to the support 2, in said distal position. The sheath 5, during the removal thereof, folds on the proximal side at its bend.

FIGS. 10 and 11 show another embodiment of the system 1, in which this system comprises a second inflatable balloon 25, fixed to the support 2 close to the stop 4, on the proximal side thereof. This second balloon 25 is supplied with inflation fluid, from outside the patient, through a conduit 26 separate from the supply conduit 16 of the balloon 3. As shown in FIG. 10, it is capable, in the inflated state, of being pressed against the inner surface of the sheath 5 so as to generate friction therewith, immobilizing the latter in the axial direction relative to the support 2. It consequently forms locking means for locking the sheath 5 in its distal position, preventing any risk of removal of that sheath during the insertion and sliding of the system 1 in the bodily conduit 100.

In practice, this second balloon 25 is in the inflated state during the insertion and sliding of the system 1 in the bodily conduit (see FIG. 10), thereby locking the sheath 5 in its distal position, and is deflated just before the deployment operation of the stent 50, so as to free the removal of the sheath 5 (see FIG. 11).

FIG. 12 shows an alternative in which the sheath 5 has a length such that it is capable of extending outside the patient when the stent 50 is opposite the implantation site. Its immobilization can therefore be done and controlled from outside the patient, using suitable immobilization means, which are released before beginning the inflation of the balloon 3.

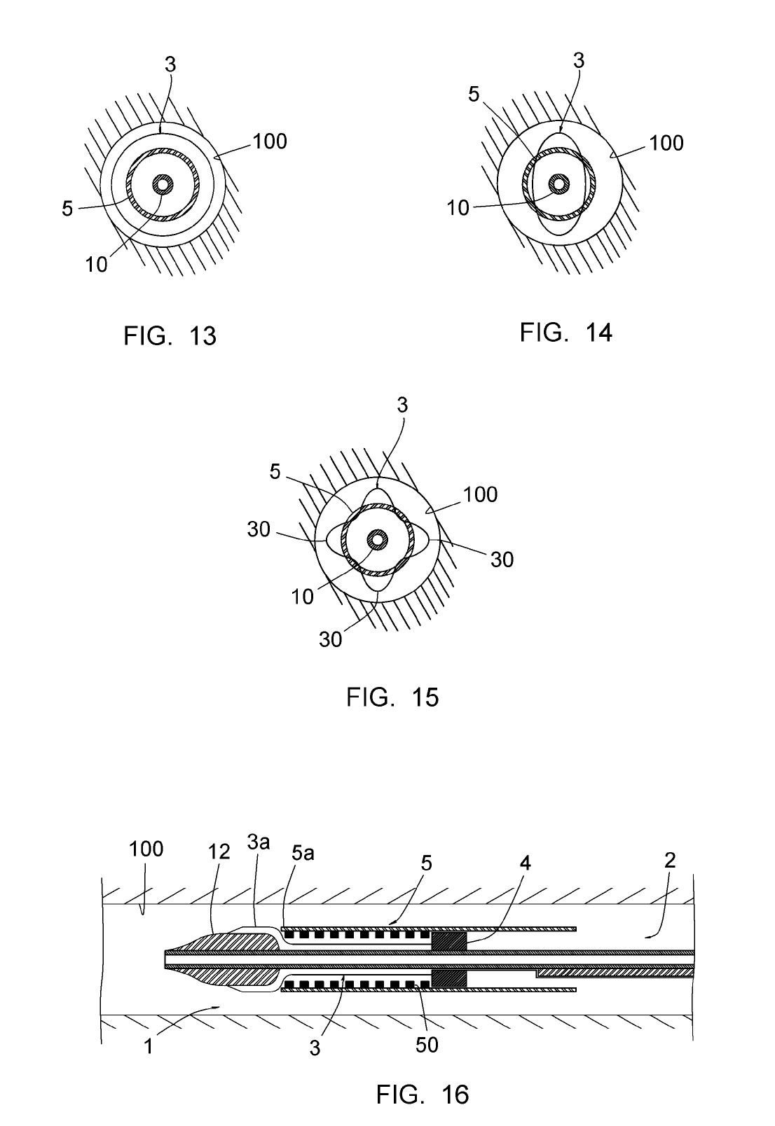

FIG. 13 shows the most frequent case of a balloon 3 assuming a circular shape in transverse section. FIG. 14 shows that it can also have an oval shape in transverse cross-section, and FIG. 15 that it can also have one or several longitudinal ribs 30, in particular four equidistant longitudinal ribs 30.

FIG. 16 shows another embodiment of the system 1, in which the distal portion 3a of the balloon 3 is dimensioned to surround a proximal part of the distal end piece 12 and is, on the distal side, connected thereto, over its entire periphery. This distal portion 3a is also shaped so as to extend, in the uninflated state, close to the distal end 5a of the sheath 5.

The balloon 3, thus shaped, allows the system 1 to have a substantially smooth and continuous outer wall between the end piece 12 and the sheath 5.

FIG. 17 shows another embodiment of the system 1, in which the balloon 3 has, in the uninflated state or the folded state illustrated, a distal portion 3a having a transverse section larger than that of the proximal portion of that balloon 3, this distal portion 3a having a more or less ovoid or hernial shape.

FIG. 18 shows still another embodiment of the system 1, in which the system includes a proximal inflation restricting element 60. This element 60 makes it possible to ensure that the inflation of the balloon 3 starts at the distal portion 3a of said balloon and progresses in the proximal direction, and to thereby prevent early proximal inflation from occurring, i.e. proximal inflation occurring at the same time as the distal inflation.

In the example shown in this FIG. 18, this restricting element 60 is in the form of a sleeve made from PTFE (polytetrafluoroethylene), surrounding the balloon 3 in an adjusted manner, having a distal end that protrudes past the distal end 5a of the sheath 5 and that is situated back from the distal end 3b of the balloon 3 in the proximal direction. This sleeve can be torn longitudinally under the effect of the inflation of the balloon 3, so as not to hinder that inflation.

The inflation of the balloon 3 is thus done from said distal end 3b and progresses in the proximal direction, the proximal portion of the balloon 3 not being able to inflate, being maintained by the sleeve; this inflation causes the progressive tearing of the sleeve from said distal end of the sleeve, which allows the inflation to continue, in the proximal direction.

As shown in FIG. 19, the sleeve can comprise a longitudinal notch 61 formed from its distal end, constituting a beginning of longitudinal tearing.

FIGS. 20 and 21 show still another embodiment of the system 1, in which said system includes an element 70 for preventing or restricting the radial expansion of the sheath 5, also resulting in preventing an early proximal inflation of the balloon 3 from occurring.

In the example embodiment shown in FIGS. 20 and 21, this element 70 comprises a sleeve made from an elastically deformable material, extending the distal end 5a of the sheath 5. This element 70 has, in the non-inflated state of the balloon 3, a radially convergent conical shape (see FIG. 20), i.e. extending from the distal end 5a to said first balloon, and expanding radially during the inflation of said balloon 3, to adopt a flared shape, radially divergent (see FIG. 21).

This radial expansion generates a radial elastic return force of the material making up said sleeve, favorable to obtaining an effective withdrawal movement of the sheath 5.

The element 70 can also comprise a ring made from a material that is not radially stretchable, fixed at the distal end 5a of the sheath 5.

FIG. 22 shows that the balloon 3 can also assume, in the inflated state, a helical groove 80, the purpose of which is to reduce the force exerted radially by the balloon 3 on the sheath 5 as well as the friction existing between this balloon 3 and the stent 50.

As appears from the preceding, the invention provides a system for delivering a stent having, relative to its counterpart systems of the prior art, the decisive advantage of allowing precise positioning of the stent in the bodily conduit to be treated.

The invention was described above in reference to embodiments provided as examples. It is of course not limited to these embodiments, but extends to all other embodiments covered by the appended claims.

What is claimed is:

1-20. (canceled)

21. A stent delivery system comprising:
a stent having a proximal end;
a support comprising the stent engaged thereon, having a longitudinal axis and being equipped with a stop against which the proximal end of the stent is applied;
a sheath having a distal end, which can be slid relative to said support and said stop, between a distal overlap position of the stent and a proximal release position for the deployment of the stent, and
a first inflatable balloon situated between said support and the stent, comprising a distal portion that protrudes past the distal end of the sheath, on the distal side thereof, when the latter is in said distal position, and having, in the inflated state, over all the length thereof or over part of the length thereof, on the distal side, a transverse section larger than or equal to that of the sheath,
the distal end of the sheath being, in said distal overlap position of the stent, situated back from said distal end, along said longitudinal axis of the support, in the proximal direction, and being situated opposite said distal portion, such that said distal portion protrudes partially past said distal end of the sheath, on the distal side of said end.

22. The system according to claim 21, wherein it includes locking means that are able to be actuated between a locking position and an unlocking position, these locking means being able, in the locking position, to immobilize the sheath in said distal overlap position and, in the unlocking position, to release the sliding of the sheath.

23. The system according to claim 22, wherein the locking means comprise a sheath having a length such that said sheath extends outside the patient when the stent is located opposite the implantation site, and means for immobilizing said sheath relative to said support, capable of being controlled from outside the patient.

24. The system according to claim 22, wherein it comprises a second inflatable balloon, fixed in position relative to said support and situated inside the sheath, the inflation of which corresponds to said locking position and the deflation of which corresponds to said unlocking position; in the inflated state, this second balloon is pressed against the inner surface of the sheath so as to generate friction therewith, immobilizing the latter in the axial direction relative to said support, and, in the deflated state, this second balloon is not pressed against the inner surface of the sheath so as to allow the sliding of the latter.

25. The system according to claim 24, wherein the said second balloon is situated close to said stop, on the proximal side thereof.

26. The system according to claim 22, wherein locking means comprise a second inflatable balloon fixed in position relative to said support and the inflation of which corresponds to said locking position while the deflation thereof corresponds to said unlocking position; this second balloon is situated outside the sheath, on the proximal side of a proximal end of said sheath; the second balloon has, in the inflated state, a diameter larger than that of the sheath, so that this second balloon forms a stop against which the proximal end of the sheath bears so as to block the sliding of the sheath; in the deflated state, this second balloon has a diameter smaller than that of the sheath, so that this second balloon does not form a stop for said proximal end of the sheath and does not hinder the sliding of the sheath.

27. The system according to claim 21, wherein the distance separating the distal end of said balloon and the distal end of the sheath when the latter is in the distal position, is comprised between one and two times the diameter of the sheath.

28. The system according to claim 21, wherein said first balloon has a cylindrical shape, with a diameter at least equal to that of the sheath.

29. The system according to claim 21, wherein said first balloon has a conical shape, whereof the distal portion, with the larger diameter, has a diameter at least equal to that of the sheath.

30. The system according to claim 21, wherein said first balloon has an oval transverse section.

31. The system according to claim 21, wherein said first balloon has one or more longitudinal ribs, in particular four equidistant longitudinal ribs.

32. The system according to claim 21, wherein said support comprises a widened distal end piece, capable of favoring the progression of the system in a bodily conduit, and the sheath comprises a sleeve extending its distal end, which is dimensioned so as to extend to said end piece, this sleeve being made from a material more flexible than that of the sheath, not offering resistance to the inflation of said distal portion of said first balloon, or a low resistance to that inflation.

33. The system according to claim 21, wherein said support comprises a widened distal end piece, capable of favoring the progression of the system in a bodily conduit, and said distal portion of the first balloon is shaped so as to surround a proximal portion of that end piece and, in the non-inflated state, to extend close to the distal end of the sheath.

34. The system according to claim 21, wherein it includes a proximal inflation restricting element, making it possible to ensure that the inflation of said first balloon starts at said distal portion of the balloon and progresses in the proximal direction.

35. The system according to claim 34, wherein the restriction element is in the form of a sleeve surrounding said balloon in an adjusted manner, one distal end of said sleeve protruding past the distal end of the sheath and being situated withdrawn from the distal end of said first balloon in the proximal direction, and this sleeve being longitudinally tearable under the effect of the inflation of this first balloon, so as not to hinder the inflation.

36. The system according to claim 35, wherein said sleeve is made from a material with a low coefficient of friction, in particular PTFE (polytetrafluoroethylene).

37. The system according to claim 35, wherein said sleeve comprises a longitudinal notch arranged from its distal end, constituting a beginning of longitudinal tearing.

38. The system according to claim 21, wherein it includes an element for preventing or restricting the radial expansion of the sheath.

39. The system according to claim 38, wherein the expansion preventing or restricting element comprises a sleeve made from an elastically deformable material, extending said distal end of the sheath, having, in the non-inflated state of said first balloon, a conical shape converging radially, i.e. extending from said distal end of the sheath to said first balloon, and expanding radially during the inflation of said first balloon, to adopt a flared shape, radially divergent.

40. The system according to claim 38, wherein the expansion preventing or restricting element can comprise a ring made from a material not radially stretchable, fixed at the distal end of the sheath.
